(19) United States
(12) Patent Application Publication
(10) Pub. No.: US 2008/0065217 A1
(43) Pub. Date: Mar. 13, 2008

(54) Artificial vertebral body

(75) Inventors: R. John HURLBERT, Calgary (CA); Lali SEKHON, Reno, NV (US); Stephan J. DUPLESSIS, Calgary (CA)

Correspondence Address: Howard M. Ellis;SIMPSON & SIMPSON, PLLC, 5555 Main Street, Williamsville, NY, 14221, US

(73) Assignee: KINETIC SPINE TECHNOLOGIES, INC., Calgary (CA)

(21) Appl. No.: 11/978,745

(22) Filed: Oct. 30, 2007

Related U.S. Application Data
(63) Continuation of application No. PCT/CA06/00675, filed on May 2, 2006.
(60) Provisional application No. 60/594,727, filed on May 2, 2005.

(51) Int. Cl. A61F 2/44

(52) U.S. Cl. 623/17.15

(57) ABSTRACT

An artificial vertebral body includes superior and inferior portions wherein said portions are positively engageable. The relative positions of the superior and inferior portions are variable about the sagittal plane. The artificial vertebral body also includes one or more means for engaging adjacent spinal structures to establish placement of the body in the spine.

### CROSS REFERENCE TO PRIOR APPLICATIONS

The present application is a Continuation of PCT application no. PCT/CA2006/000675, filed May 2, 2006, which claims priority from U.S. application No. 60/594,727, filed May 2, 2005. The entire disclosures of these applications are incorporated herein by reference.

### FIELD OF THE INVENTION

The present invention generally relates to devices and surgical methods for the treatment of various types of spinal pathologies. More specifically, the present invention is directed to vertebral body replacements and procedures for performing such replacements.

### BACKGROUND OF THE INVENTION

The spine has four natural curves; two are lordotic and two are kyphotic. The cervical and lumbar curves are lordotic, while the thoracic and sacral curves are kyphotic. While these curves of the spine help to distribute mechanical stress as the body moves, conditions may develop where there are extreme curvatures. For example, while the upper or thoracic region of the spine is normally curved forward, if the curve exceeds 50°, it is considered abnormal or “kyphotic”. Lordosis is an abnormal increase in the normal lordotic curvature of the lumbar spine; excessive lordosis may cause an extreme inward curve in the lower back.

The techniques, instrumentation and implants for treating conditions or abnormalities of the spine have adapted to address many forms of spinal injury and deformities that can occur due to trauma, disease or congenital effects. One type of spinal deformity, a kyphosis, involves a prolapse of the vertebral column towards the front of the body, often caused by the destruction of the vertebral body itself.

Several events can distort the spine leading to conditions like accentuated kyphosis or hyper-lordosis. Because the natural tendency of the spine is to curve, a weakness in any component thereof or the supporting structures may lead to such conditions. For example, a diseased thoracic vertebra will ordinarily crumble its forward edge first, increasing the kyphotic curve. Conditions that can do this include cancer, tuberculosis, Scheuermann's disease, and certain kinds of arthritis. Healthy vertebra can fracture forward with rapid deceleration injuries, such as in car crashes. Osteoporosis may also contribute to such conditions. As result of any of these conditions and their underlying etiology, it may be necessary to consider vertebral body replacement.

Where it is necessary to replace at least a portion of a vertebral body for the reasons noted above, previous techniques have involved reconstruction of that portion of the vertebral body with a polymerizable paste or a bone graft which is frequently modelled to give it the shape of intact vertebral body. Frequently, autologous bone, such as that extracted from the ilium, is used to bridge the space. The polymerizable paste can include a PMMA bone cement. Various artificial apparatus have also been developed to address structural failure of various parts of the spinal column.

While there is a need for the replacement of injured and/or diseased vertebral bodies, which cause, or are a result of, various spinal diseases, previous apparatuses and techniques have, however, several drawbacks. They have been fashioned to provide support between adjacent vertebral bodies by creating a fusion across the diseased segment thereby eliminating movement in the spinal column. In addition to reducing range of movement in the spine this can also cause premature degeneration of spinal joints above and below the fusion site. They also require additional instrumentation at the front or back of the spine to secure them in place.

The present invention, in one aspect, provides an artificial vertebral body that obviates or mitigates at least some of the deficiencies of previous apparatuses and techniques.

### SUMMARY OF THE INVENTION

In one aspect, the invention provides an vertebral body for replacing a naturally occurring a vertebral body in a spine.

Thus, in one aspect, the invention provides an artificial vertebral body comprising:

a superior portion and an inferior portion, each of the superior and inferior portions having superior and inferior surfaces and lateral sides;

the superior surface of the inferior portion being in contact and in positive engagement with the inferior surface of the superior portion;

the superior surface of the superior portion and the inferior surface of the inferior portion having one or more engagement means to engage adjacent spinal structures.

### BRIEF DESCRIPTION OF THE DRAWINGS

Various objects, features and attendant advantages of the present invention will become more fully appreciated and better understood when considered in conjunction with the accompanying drawings, in which like reference characters designate the same or similar parts throughout the several views.

FIGS. 1(a) to (g) show various views of embodiments of the present invention.

FIG. 2 shows a side view of an embodiment of the present invention.

FIG. 3 shows a perspective view of an embodiment of the present invention.

FIGS. 4(a) to (d) show various views of embodiments of the present invention.

FIGS. 5(a) to (c) show various views of embodiments of the present invention.

FIGS. 6(a) to (c) show various views of embodiments of the present invention.

FIG. 7 shows side views of an embodiment of the present invention.

FIG. 8 shows side views of an embodiment of the present invention.

FIG. 9 shows side view of an embodiment of the present invention.

### DETAILED DESCRIPTION OF THE INVENTION

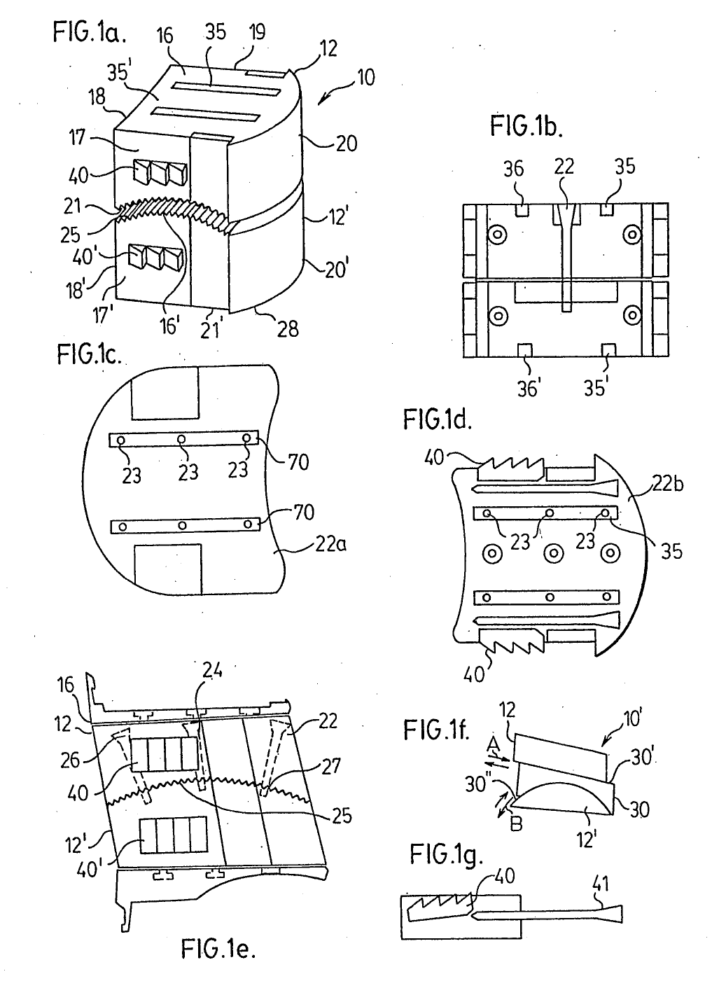

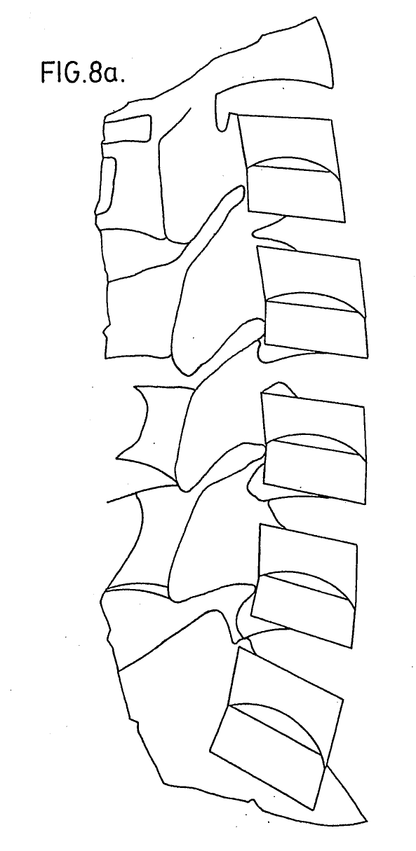

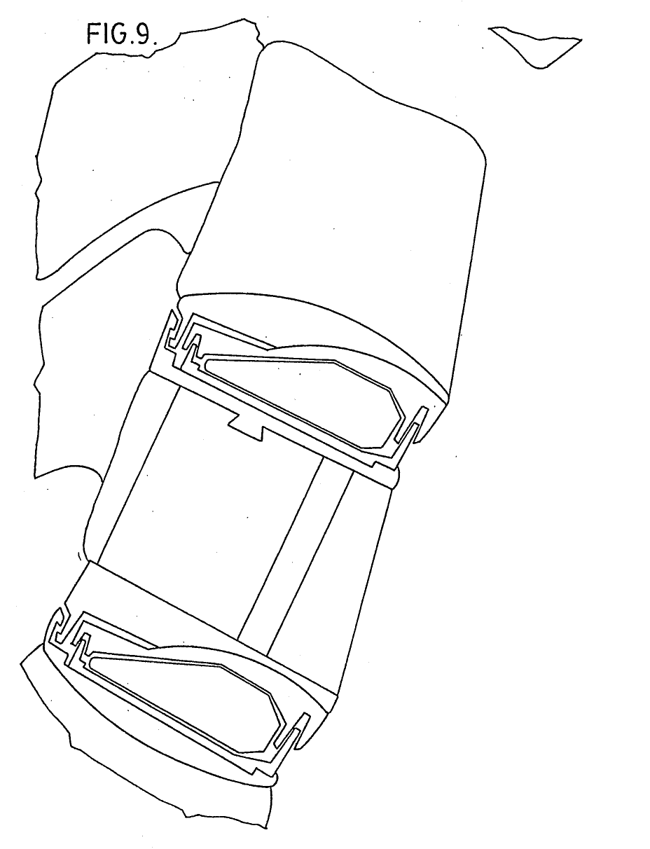

In order that the invention may be more fully understood, it will now be described, by way of example, with reference to the accompanying drawings in which FIGS. 1 through 9 illustrate embodiments of the present invention.

In the description and drawings herein, and unless noted otherwise, when discussing anatomical plans of view, it will be understood that the terms “front” and “back” shall be used to refer to the front and back in the coronal or frontal plane. The terms “left” and “right” shall be used to refer to left and right in the sagittal or lateral plane. The terms “up” and “down” shall be used to refer to up and down in the axial transverse. It will be understood that a reference to “medial” shall refer towards the midline of a body. It will be understood that a reference to “lateral” shall refer to away from the midline of a body. It will be understood that a reference to “inferior” shall refer to lower, below or down and “superior” shall refer to upper, above or up. It will be further understood that a reference to “anterior” shall refer to front and “posterior” shall refer to the rear or back.

The present invention provides an artificial vertebral body that can be used to replace at least a portion of vertebral bodies in different regions of the spinal column or, alternatively, the entire vertebral body or corpus (e.g. the drum shaped structure at the anterior end of the vertebrae) can be replaced. Specifically, different embodiments of the vertebral body according to the present invention may be used in the lumbar, thoracic, and cervical spine regions.

FIG. 1(a) illustrates an artificial vertebral body 10 in accordance with an embodiment of the present invention. Vertebral body 10 has a superior portion 12 and an inferior portion 12′. As shown in FIG. 1(e), portions 12 and 12′ have a generally wedged, trapezoid shape to approximate configuration of a normal vertebral body in a lordotic spine. As shown in FIG. 1(a), the superior element 12 includes an upper or superior surface 16, a lower inferior surface 21, anterior and posterior surfaces 20 and 18, respectively and left and right lateral side surfaces 19 and 17, respectively. Similar surfaces are provided on portion 12′ except identified as 16′, 17′, 18′, 19′, 20′ and 21′, as shown in FIGS. 1 and 3. Anterior surface 20 of portion 12 may extend beyond the anterior surface 20′ of portion 12′, the function of which will be described below.

Portions 12 and 12′ are affixed together by at least one fastener. As shown in FIGS. 1(b) and (e), there are three fasteners in the form of screws 22, 24 and 26.

Anterior surface 20 is generally convex, while inferior surface 21 is generally concave. Posterior surface 18 is also concave. The anterior surface or side 20, after insertion into the spinal column, is generally facing anteriorly, namely facing towards the front of the body, while the posterior side 18 is positioned towards the spinal cord contained within the spinal column (towards the back of the body). The superior surface 16 of the vertebral body may be of smaller dimensions than the inferior surface 21. Similarly the superior surface 16′ may be of smaller dimensions that the inferior surface 21′, causing asymmetry between upper and lower surfaces, more closely approximating normal anatomical relationships in the cervical spine. This asymmetry may be less pronounced or reversed in cases where the invention is for use in the thoracic or lumbar spine regions.

Portion 12 is provided with overhang 28 of the convex surface 20 with respect to the sides of vertebral body. Overhang on the sides of the anterior (or front) curved surface of the vertebral body produces an edge of approximately 90 degrees to the true lateral wall of the vertebral body. Portion 12′ is similarly shaped. Over hang 28 prevents posterior migration of portion 12 into the spinal cord upon insertion thereof into a surgical vertebrectomy defect. A smooth anterior surface may also reduce postoperative dysphagia by reducing adhesions between the implant and the posterior pharyngeal wall.

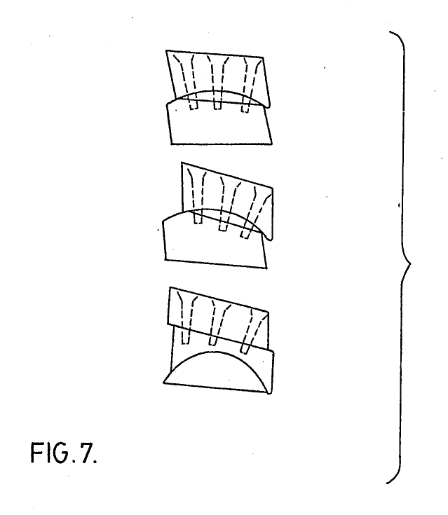

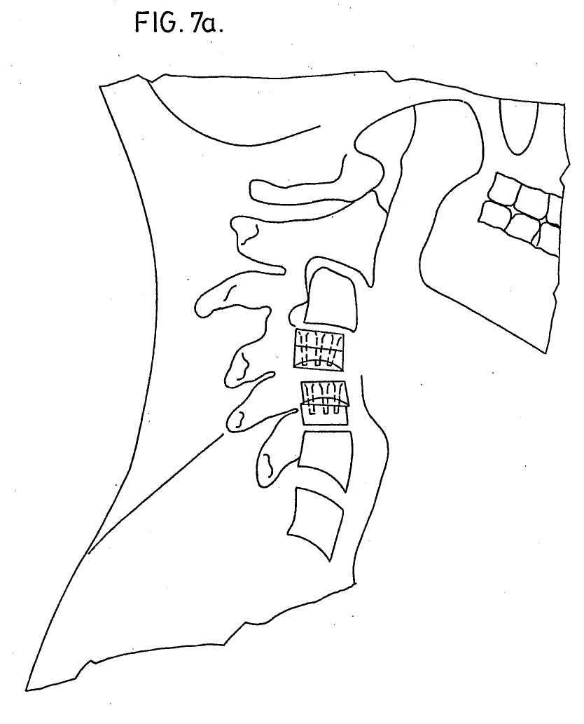

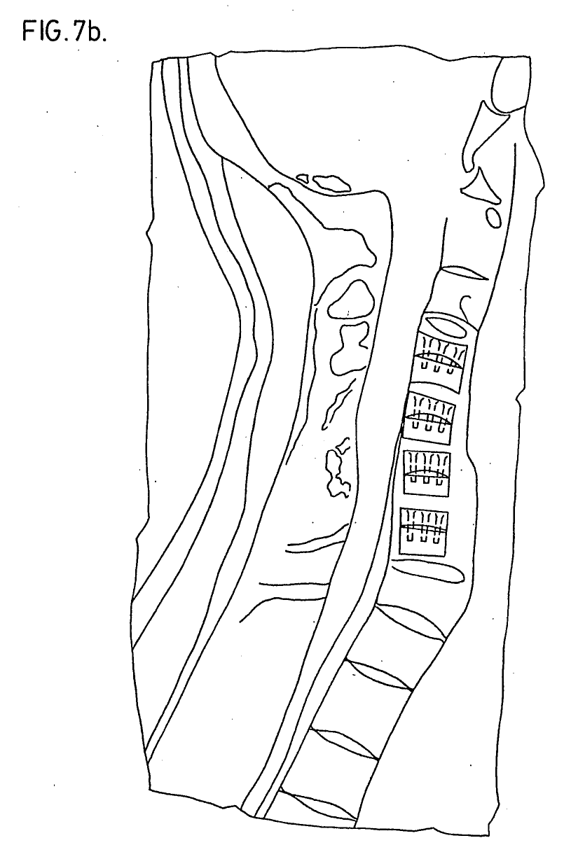

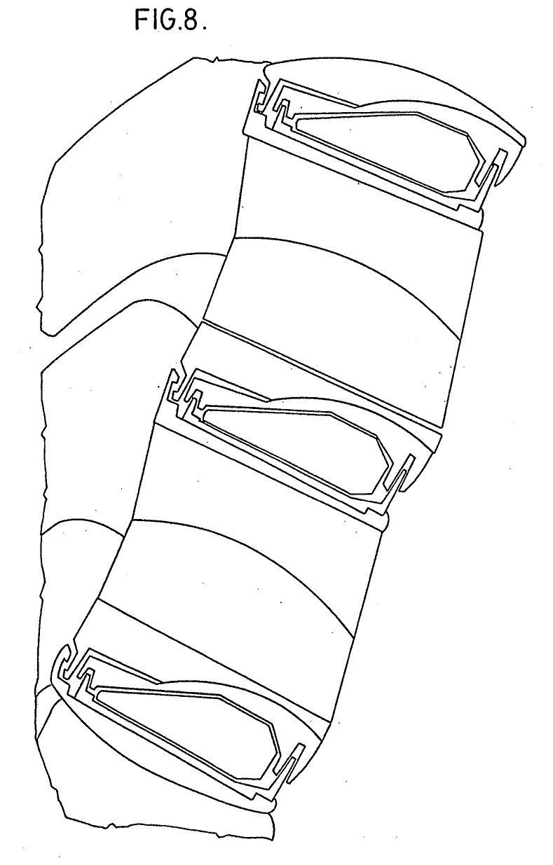

Portions 12 and 12′ are joined by way of a toothed or textured curved locking mechanism. Such mechanism is formed with the inferior surface of portion 12 and the superior surface of portion 12′ having complementary toothed surfaces. In this manner, when the portions 12 and 12′ are positioned together, the toothed surfaces engage to prevent further movement between such surfaces. This locking mechanism is tightened by at least one of the fasteners affixing portions 12 and 12′ together. In one embodiment of the present invention, there is provided, as shown in FIGS. 1(b) and (e), screws 22, 24 and 26 recessed into the midline of the superior surface of portion 12. Loosening of the locking mechanism allows adjustment of the angular relationship of upper portion 12 to the lower portion 12′ of vertebral body 10. In such a manner, upper surface 16 of vertebral body 10 can be made parallel to lower surface 21′ or the two surfaces can be angled or off-set with respect to each other. FIG. 1(f) shows how surfaces can be offset. In addition, FIGS. 7 to 9 show how the portions can be off set. This accommodates angular variations of lordosis and kyphosis amongst different areas of the spine and variations in the spines of different people.

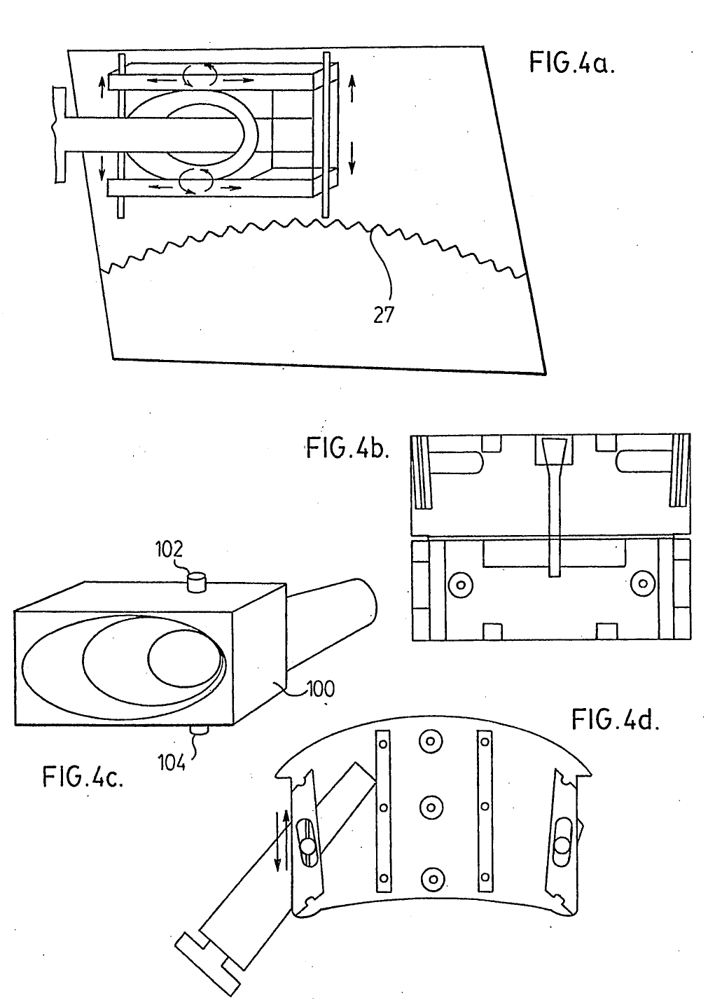

In one embodiment, the locking mechanism consists of adjustment fasteners 22, 24 and 26 provided generally in the midline of portion 12 and recessed into superior surface 16 of portion 12. As seen in FIG. 1(e), a locking plate 25 is provided between portions 12 and 12′ and is positioned adjacent to the convex curve of the inferior portion 12′ of the vertebral body. The ends of screws 22, 24, and 26 engage locking plate 25. Locking plate 25 receives the screws 22, 24 and 26 and acts like a “blind nut”, which receives a screw that can be subsequently tightened without the nut spinning. Tightening screws 22, 24 and/or 26 into locking plate 25 cinches it up against the inside of 12′ which in turn then engages against the teeth 27 of toothed or textured curved surface 21 of portion 12. Teeth 27 are shown in greater detail in FIG. 4(a). Pressure between the screw heads 22, 24, and 25 against portion 12, and pressure from the locking plate against portion 12′ keep the teeth engaged between 12 and 12′ preventing the vertebral body from changing shape. Locking plate 25 may be sufficiently wide or as sufficiently long so as to fit within portion 12′. Locking plate 25 is provided against toothed or textured curved surface 21 so as to allow angular motion of portion 12 between angled posterior and anterior positions which allows for the adjustment of the angular relationship of upper portion 12 to the lower portion 12′ of vertebral body 10. As will be understood, such adjustment is made by loosening screws 22, 24 and 26, disengaging the toothed surfaces of the portions 12 and 12′ and moving such portions relative to one another to achieve the desired placement.

As shown in FIG. 1(e), superior surface 16′ of lower portion 12′ is adapted to mate with the curved inferior surface 21 of upper portion 12. The mating of surface 16 and surface 21 provided for an interface that separates the upper portion 12 of vertebral body 10 from lower portion 12′. Changes in angle necessitate a degree of translation of the upper half with respect to the lower half of the artificial vertebral body.

In a further embodiment of the present invention, as shown in FIG. 1(f), the artificial vertebral body of the invention may include a third portion 30 provided between the portions 12 and 12′ in order to produce pure translational adjustments of portion 12 of the vertebral body 10′. As shown in FIG. 1(f), the third portion 30 provides an additional interface between the portions 12 and 12′. As shown in FIG. 1(f) and FIG. 7, it will be appreciated that the addition of portion 30 allows the creation of a more kyphotic angle in the vertebral implant without the large degree of offset required in the “two component” version. As shown in FIG. 1(f), the radius of the curvature is less with the embodiment encompassing three portions 12, 12′ and 30, making the curve sharper than in the two piece design. It will be understood, however, that the radius can be varied depending on the application. Similarly the dimensions of the three components shown in 10′ (FIG. 1(f)), namely portions 12, 12′ and 30 may also be varied in length, width, and height.

Third portion 30 has an upper or superior surface 30′ and a lower or inferior surface 30″, which engage with surfaces 21 and 16′ respectively. Upper surface 30′ is generally flat, while lower surface 30″ is generally curved to mate with superior surface 16′ of portion 12′. It will be understood in such an embodiment that inferior surface 21 of portion 12 would be need to be configured to mate with surface 30′. This allows the artificial vertebral body to take on a more skewed and kyphotic shape to treat conditions of more severe malalignment. In this embodiment, vertebral body 10′ consists of three elements or portions 12, 12′ and 30. These elements are affixed by two sets of locking mechanisms similar to those described above through toothed or textured surfaces that interact to form two interfaces. One portion of body 10 translates backwards or forwards on a middle portion. As provided in FIG. 1(e), portion 12 can move anteriorly or posteriorly as shown by arrows A. Another portion of body 10 angles backwards and forwards on the middle section as well. As provided in FIG. 1(e), portion 30 can move as provided by arrows B. Adjustment screws are recessed from the exposed upper and lower surfaces of the body lock the pieces together in their desired configuration.

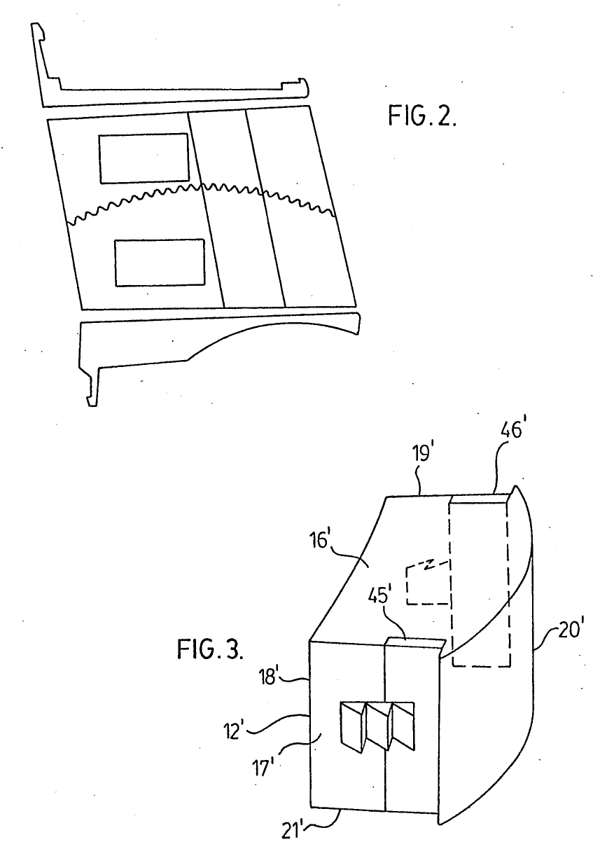

As shown in FIGS. 1(e) and 2, the artificial vertebral body of the present invention can integrate or co-operate with an artificial intervertebral disc such as a disc that is described in applicants' co-pending application No. 60/594,732 (the entire contents of which is incorporated herein by reference). As described in such application, an artificial disc is provided with one or more “stabilising keels” on at least one of its outer surfaces. As shown in FIGS. 1(a), (b), and (d) there are provided openings 35 and 36 on surface 16 as well as opening 35′ and 36′ on lower surface 16′. Openings 25, 36 as well as 35′ and 36′ contain fastener openings through which screws extending from endplates of an artificial disc or the like can be secured, fastening the disc endplate tightly to the vertebral body. The keels of the artificial disc endplate fit into the openings so as to align the screw conduits.

FIGS. 1(c) and 1(d) demonstrate adjacent or integrating surfaces of disc endplate (see FIG. 1(c)) with artificial vertebral body (see FIG. 1(d)). Keels 70 provided on the artificial disc endplate insert into slots 35 and 36 (or 35′ and 36′) of the vertebral body. Screw holes allow screws to be inserted, attaching the endplates 22a and 22b to the artificial vertebral body in multilevel disc and body reconstruction. Screw receptacles 23 are shown in the artificial vertebral body diagrams as well as on the keels 70 of the artificial disc. FIG. 1(c) is a diagram of the surface of the artificial disc endplate that “flips” over and integrates with the vertebral body shown in 1(d). The keels 70 of the disc endplate fit into troughs 35, 35′, 36, and 36′ of the artificial vertebral body. In one embodiment, screws fix superior endplate 22b to inferior surface 16′ of the vertebral body, and the inferior endplate 22a of the disc to superior surface 16 of the vertebral body. The screws are inserted from the inside surface of the disc endplates—i.e. the disc must be disassembled to attach the endplates to the artificial vertebral body,

As shown in FIGS. 1(a) and (d) as well as FIG. 3, there are provided two sets of stabilizing fins 40 and 40′ located on each lateral surface of portions 12 and 14 of the vertebral body. Fins 40 and 40′ move from a recessed position to an extended position by means of fin set screws, such as fin set screw 41 as shown in FIG. 1(g). Insertion of vertebral body 10 into the surgical vertebrectomy defect occurs with fins 40 and 40′ in a recessed position. Once the vertebral body 10 is inserted in the correct position, fin set screws, such as fin set screw 41 can be tightened causing individual fins of fin sets 40 and 40′ to push out from their recessed position in artificial vertebral body 10 engaging surrounding bone and securing vertebrae 10 in place against the remaining native bone inside the patient.

The individual fins of fin sets 40 and 40′ are designed to act against vertebral body extrusion. They are tapered toward their posterior aspect and that of the artificial vertebral body but angled perpendicular to the body along their anterior surface. They act in opposition to the overhang of the curved front surface of the artificial vertebral body, which prevents posterior migration.

As shown in FIG. 3, porous reservoirs 45′ and 46′ of portion 12′, as well as 45 and 46 of portion 12 (not shown) are located behind curved surface 20 or 20′ along lateral surfaces 19 and 21 as well as 19′ and 21′ of portions 12 and 12′ respectively. Reservoirs 45, 46, 45′ and 46′ act as hollow cages with small perforations in their outside walls. The cages are open at their upper and lower ends to allow for insertion of a substance to promote the growth of bone. The porous reservoirs contain this bone growth substance helping to release it locally in a controlled fashion encouraging bone to actively grow into the perforations and stabilize the artificial vertebral body against normal bone inside the spine of the recipient patient. The porous reservoirs can rest adjacent to similar reservoirs located in the artificial disc endplate. It will be understood that the exterior surfaces of the artificial vertebral body 10 or 10′ may also include various physical features such as a porous or pitted surface, a plurality of pins, ribs etc. that promote bony in-growth so as to anchor the prosthesis in place in the spine. Various other such anchoring means will be known to persons skilled in the art.

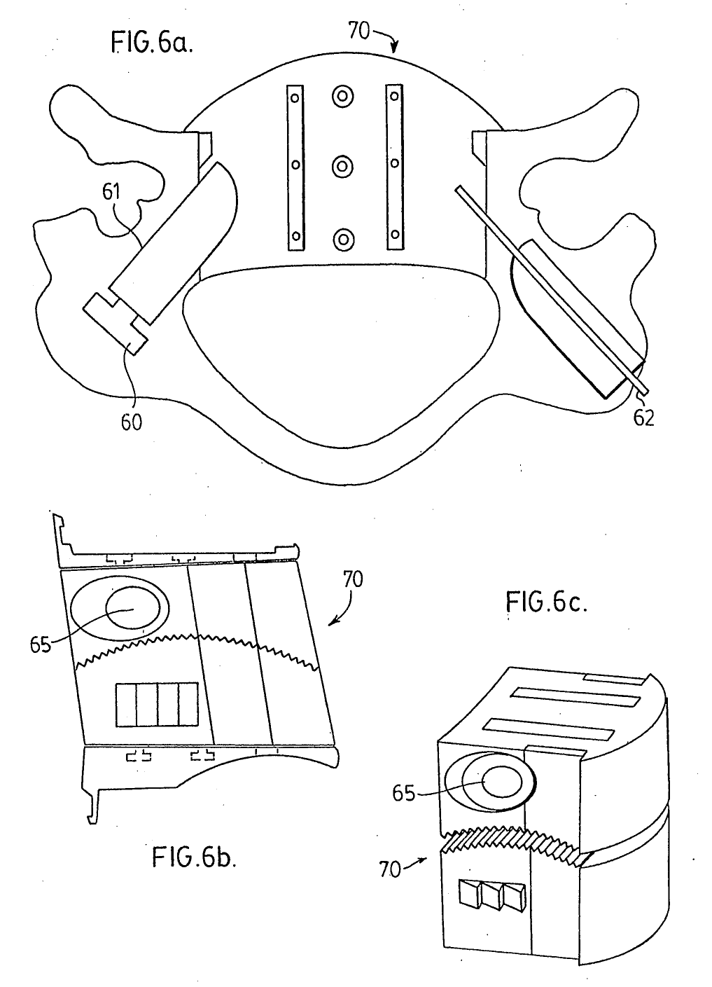

In another embodiment, sets of stabilizing fins 40 and 40′ and/or reservoirs 45, 46, 45′ and 46′ in one portion of the vertebral body 10 might be partially or completely substituted for a depression or indentations, as shown in FIGS. 4, 5 and 6. These depressions serve as insertion points for artificial pedicles that are adapted to be received within the depressions. The pedicle is the part of each side of the neural arch of a vertebra. It connects the lamina with the vertebral body. In one embodiment of the present invention, the artificial pedicle might be hollow to allow a drill to be positioned inside of it, and used to drill through the depression into the body of the artificial vertebrae. A screw might then be inserted through the pedicle tightening into the artificial vertebral body and securing the pedicle against the artificial vertebral body. As shown in FIG. 6(a), there is provided pedicle fastener 60 in artificial pedicle 61, which rests in depression or aperture 65 of vertebral body 70 (see also FIGS. 6(b) and (c)). As can also be seen in FIG. 6(a) a drill bit 61 advances into a lateral surface of vertebral 70.

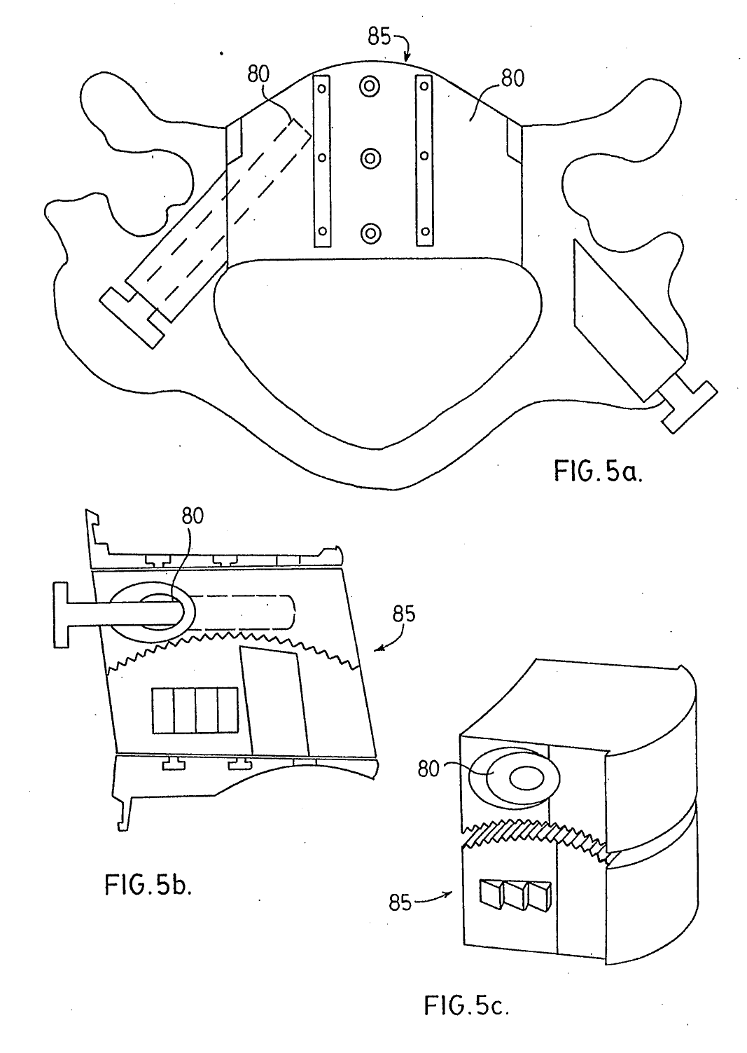

In yet a further embodiment, as shown in FIG. 5, the aperture or depression might extend farther into a portion of the vertebral body forming a pedicle fastener receptacle or sleeve that doesn't require drilled as shown in FIG. 6. As shown in FIGS. 5(a), (b) and (c), receptacle 80 is angled tangential to the side of vertebral body 85 to generally cross from one lateral surface towards the anterior surface or towards the midline of the front of the artificial vertebral body. Receptacle 80 may have threads in its walls to receive a threaded fastener of appropriate dimensions. The receptacle or pedicle screw sleeve can be embodied by a hollowed-out portion of the vertebral body or may be fashioned as a separate component, assembled into the artificial vertebral body as shown in FIG. 5.

As shown in FIGS. 4(a) to (d), a receptacle or pedicle screw sleeve 100 can be fashioned as a separate component and assembled into the artificial vertebral body. Posts 102 and 104 arising from the upper and lower surfaces of the sleeve can act as a hinge recessed inside the artificial vertebral body (as shown in FIG. 1(d)) so that the pedicle sleeve 100 can rotate about post. This allows the pedicle screw sleeve 100 to accommodate pedicle screws from different angles (as shown in FIG. 1(d)). The outer end of the pedicle screw receptacle is expanded so that an artificial pedicle can be inserted into it, locking it in place with respect to the receptacle and the artificial vertebral body.

The posts arising from the upper and lower surfaces of the pedicle screw receptacle can be embedded in recessed grooves running front to back in the artificial vertebral body. The grooves provide a track along which the pedicle sleeve can move forwards or backwards in relation to the artificial vertebral body while maintaining angular motion about the posts.

The grooves can be angled inwards or outwards (front to back) to prevent forwards or backwards migration of the artificial pedicles once they have been secured to the artificial vertebral body. It is envisioned that the artificial pedicles will be directly or indirectly connected to each other from behind in addition to their connection to the artificial vertebral body. The additional connection prevents the artificial pedicles from sliding along their posts inside grooves on either side of the vertebral body that point in opposing directions.

Additionally, a separate rectangular compartment built into the wall of the artificial vertebral body may house the grooves containing the pedicle screw sleeve. This rectangular housing could be connected to the artificial vertebral body by two side rails protruding into it at each end. The side rails (tongue in groove) prevent the housing from extruding outside or inside of the artificial vertebral body but allow the housing to move upwards and downwards within the wall of the artificial vertebral body. This is illustrated in FIGS. 4b and 4d.

The side rails can be angled in towards the middle of the artificial body or out towards the sides (from the top downwards) to prevent the rectangular compartment from moving once the artificial pedicles have been secured to the artificial vertebral body. It is envisioned that the artificial pedicles will be directly or indirectly connected to each other from behind in addition to their connection to the artificial vertebral body. The additional connection prevents the artificial pedicles and their respective rectangular housings from sliding up or down on rails that point in opposing directions on either side of the artificial vertebral body.

The artificial vertebral body can be used with artificial discs to reconstruct multiple levels in the spinal column. It can be manufactured in a variety of widths, heights, and depths.

Although the invention has been described with reference to certain specific embodiments, various modifications thereof will be apparent to those skilled in the art without departing from the purpose and scope of the invention as outlined herein. The entire disclosures of all references recited above are incorporated herein by reference.

We claim:

1. An artificial vertebral body comprising:
a superior portion and an inferior portion, each of said superior and inferior portions having superior and inferior surfaces and lateral sides;
the superior surface of said inferior portion being in contact and in positive engagement with the inferior surface of said superior portion;
the superior surface of said superior portion and the inferior surface of said inferior portion having one or more engagement means to engage adjacent spinal or artificial spinal structures.

2. The vertebral body of claim 1 wherein said positive engagement is provided by one or more engagement means.

3. The vertebral body of claim 2 wherein said engagement means comprises the superior surface of said inferior portion and the inferior surface of said superior portion having cooperatively toothed surfaces.

4. The vertebral body of claim 3 wherein said engagement means further comprises one or more screws extending from one of said portions to the other of said portions.

5. The vertebral body of claim 4 wherein the relative position of the superior and inferior portions about the sagittal plane may be varied by releasing and re-engaging the engagement means.

6. The vertebral body of claim 5 wherein the anterior surface of said body is convexly curved about the coronal plane.

7. The vertebral body of claim 6 wherein the posterior surface of said body is generally flat.

8. The vertebral body according to claim 1 wherein said body includes outwardly extending fins to engage adjacent bony structures.

9. The vertebral body of claim 8 wherein said fins are extendable.

10. The vertebral body of claim 9 wherein said body includes one or more adjustment screws to extend said fins.

11. The vertebral body of claim 10 wherein said fins are provided on lateral sides of said body.

12. The vertebral body of claim 11 wherein one or more external surfaces of said body include one or more physical and/or chemical bone growth promoters.

13. The vertebral body of claim 12 wherein said one or more external surfaces includes a reservoir for holding and releasing one or more bone growth promoting compounds.
